(19) United States
(12) Patent Application Publication
(10) Pub. No.: US 2009/0202572 A1
(43) Pub. Date: Aug. 13, 2009

(54) COMPOSITIONS AND METHODS FOR MODULATING BONE MASS

(76) Inventors: Gerard Karsenty, New York, NY (US); Bruce Devens, Oakland, CA (US)

Correspondence Address: DLA PIPER LLP (US), 4365 EXECUTIVE DRIVE, SUITE 1100, SAN DIEGO, CA, 92121-2133, US

(21) Appl. No.: 11/913,168

(22) PCT Filed: May 13, 2006
(86) PCT No.: PCT/US2005/016929

§ 371 Date: Aug. 27, 2008

Related U.S. Application Data
(60) Provisional application No. 60/571,558, filed on May 14, 2004.

(51) Int. Cl. A61K 39/395; C07C 215/60; C07D 285/10; C07C 217/72; C07K 7/06; C07K 16/00; A61K 31/138; A61K 31/137; A61K 31/433; A61K 38/22; A61P 19/10

(52) U.S. Cl. 424/178.1; 564/360; 548/134; 564/348; 530/329; 530/391.7; 514/651; 514/653; 514/362; 514/12

(57) ABSTRACT

The instant invention relates to compositions and methods for treating or preventing bone diseases. In certain aspects, the invention provides compositions comprising a β-adrenergic antagonist or agonist associated to a bone-targeted molecule, as well as methods of modulating bone mass and/or growth in a mammal by administering a composition of the present invention. In other aspects, the invention provides methods of modulating bone mass and/or growth in a mammal by administering a composition comprising a β2-selective antagonist or agonist.

### BACKGROUND OF THE INVENTION

Bone constantly remodels itself throughout the life of an individual, removing old bone and replacing it with new bone. This remodeling process is carried out through two well-defined cellular processes. Resorption of preexisting bone is mediated by osteoclasts, and de novo bone formation by osteoblasts. An imbalance in remodeling leads to osteoporosis, a disease characterized by low bone mass with microarchitechtural deterioration leading to increased fragility. Specifically, relatively increased bone turnover and enhanced osteoclastic activity at the expense of osteoblastic activity underlies osteoporosis. This can be caused by a variety of factors, including postmenopausal estrogen depletion, drug therapies such as glucocorticoids, transplantation and other unrelated diseases that influence bone turnover.

Osteoporosis is estimated to affect 200 million women worldwide, and often leads to immobility and in some cases death. A Physiological hallmark of osteoporosis is lowered bone mass which renders the bone susceptible to fractures. Osteoporosis and other diseases of bone and cartilage are responsible for a significant portion of healthcare expenditures in developed countries—US $14 billion is spent annually on treating osteoporotic fractures in the U.S. alone (Dewitt, Nature 423: 314-15, 2003). Current treatments for osteoporosis mainly retard, but do not completely reverse, bone mineral density loss.

It is thus desirable to have methods and compositions to treat bone diseases by increasing bone mass. Such methods and compositions are provided herein.

### SUMMARY OF THE INVENTION

The present invention provides conjugated drugs for regulating bone growth and bone density. Generally, the compounds of the invention are conjugated drugs including a β-adrenergic agent associated with a bone-targeting moiety, wherein the latter increases local delivery and/or efficacy of the β-adrenergic agent to osteoblasts relative to the β-adrenergic agent alone.

As described in more detail below, the β-adrenergic agent and bone-targeting moiety are covalently associated, or can be non-covalently associated.

One benefit to certain of the subject conjugates is to have a therapeutic index with respect to unwanted side-effects, e.g., effects resulting from adrenergic antagonism or agonism in other parts of the body, which is greater than the therapeutic index of the β-adrenergic agent alone.

In certain preferred embodiments, the conjugated drug is represented in the general formula (I):

(A)ₘ*(B)ₙ

wherein
A, independently for each occurrence, represents a β-adrenergic agent;
B, independently for each occurrence, represents a bone-targeting moiety;
n and m each independently represent integers of 1 or greater; and
* denotes a covalent or non-covalent interaction associating the β-adrenergic agent(s) A with the bone-targeting moieties B.

In certain embodiments, the associating interaction between the A and B moieties can be reversible or metabolized under physiological conditions in which the conjugated drug has been distributed and/or localized to bone, e.g., the dissociation releasing A or a prodrug form of A.

In other embodiments, the associating interaction between the A and B moieties is irreversible, e.g., the β-adrenergic agent retains, with respect to osteoblasts, β-adrenergic activity in the conjugated form.

Those skilled in the art will appreciate that the conjugated drugs of the present invention include embodiments in which the β-adrenergic agent is a β-adrenergic antagonist, and other embodiments in which the β-adrenergic agent is an agonist.

In certain embodiments, the subject conjugated drugs can be used as part of a method for increasing anabolic bone growth and/or bone density in a mammal, e.g., a human patient, companion pet and/or livestock.

In other embodiments, the subject conjugated drugs can be used as part of a method for decreasing anabolic bone formation in a mammal, e.g., a human patient, companion pet and/or livestock.

Still another aspect of the invention provides a packaged pharmaceutical comprising a conjugated drug of the present invention in a form suitable for use in human patients, and associated with instructions and/or a label instructing appropriate use and side effects of the conjugated drug in the treatment or prophylaxis of a bone disease.

### BRIEF DESCRIPTION OF THE DRAWINGS

FIG. 1 shows increased bone formation induced by Adrb2 deficiency. (a) von Kossa staining of vertebral sections. Six month old Adrb2−/− and Adrb2+/− mice display an increase in bone volume over tissue volume (BV/TV) compared to wt littermates. (b) Bone formation parameters: bone formation rate (BFR), osteoblast surface over bone surface (ObS/BS) and osteoblast number over bone perimeter (ObNb/BPm) are increased in Adrb2−/− and Adrb2+/− mice. (c) Bone resorption parameters: osteoclast surface over bone surface (OcS/BS), osteoclast number over bone perimeter (OcNb/BPm) and urinary elimination of deoxypiridinoline (dpd) are decreased in Adrb2−/− and Adrb2+/− mice. In contract propranolol (PRO) treated wt mice do not display a significant decrease in bone resorption parameters. n=8, *:p<0.05.

FIG. 2 shows that the SNS acts on osteoblasts to regulate bone resorption. (a) In vitro osteoclastogenesis is not affected by Adrb2 deficiency. BBMs were differentiated with the indicated amounts of RANK-L and MCS-F and the number of TRAP+ osteoclasts was counted after 5 days. (b) In vitro osteoclast differentiation is not affected by Isoproterenol (ISO) treatment. BBMs were differentiated in presence of MCS-F and RANK-L with or without 10 uM ISO and the number of TRAP+ osteoclasts was counted after 5 days. (c) ISO treatment does not induce cAMP production in mature osteoclasts. BBMs were differentiated in presence of MCS-F and RANK-L and were treated by ISO (10 μM), dobutamine (Dobu, 10 μM) or calcitonin (100 pg/ml). Intracellular cAMP production was measured by EIA. (d) ISO stimulated osteoclast differentiation via stimulation of b2AR in osteoblasts. Osteoblasts and BMMs were co-cultured with 1,25(OH)2-vitamin D (10-8 M) with or without ISO (10 uM) and the number of TRAP+ osteoclasts was counted after 4 days. (e) ISO induced Rank-l expression in osteoblasts, via b2AR. (f) ISO induced IL6 expression in osteoblasts, via b2AR. WT and Adrb2 primary osteoblasts were treated for the indicated time with ISO (10 μM) and gene expression was quantified by real-time RT-PCR. (g) Schematic diagram of the structure of Rank-1 and IL6 promoters. Boxes represent CREB-like consensus binding sites.

FIG. 3 shows that Isoproterenol (ISO) treatment leads to increased expression of RANK-L and IL6.

FIG. 4 shows protective effect of b2-adrenergic receptor deficiency against ovariectomy-induced bone loss.

FIG. 5 shows that Isoproterenol (ISO) and Parathyroid hormone (PTH), but not dobutamol, stimulate cAMP production in osteoblasts.

### DETAILED DESCRIPTION OF THE INVENTION

### I. Overview

The present invention features compositions for bone-targeted delivery of a β-adrenergic antagonist and agonists (collectively herein “β-adrenergic agents”) and methods of using such compositions to modulate bone density and growth. In general, the compositions of the present invention provide β-adrenergic agents that are associated, covalently or non-covalently, with one or more moieties (herein “bone-targeting moieties”) that enhance distribution and/or localization of the β-adrenergic agent to bone and other osteoblast-containing organs/compartments.

As described in this application, Applicants identified sympathetic signaling as a key regulator of bone resorption through its ability to regulate in osteoblasts the expression of several genes favoring osteoclast differentiation. This discovery along with previous observations indicates that the sympathetic nervous system (SNS) is a central regulator of bone remodeling that ultimately favors bone loss. The down-regulation of bone formation coupled with the up-regulation of bone resorption by the SNS is unique among all the known physiological regulators of bone remodeling. It is also demonstrated that these two functions need not be always co-regulated in the same direction. Further, the observation that haploinsufficiency at the Adrb2 locus has such profound consequences on bone remodeling also underscores the importance of sympathetic signaling in the control of bone mass.

It has previously been described that osteoblasts express β-adrenergic receptors, and that β-adrenergic agents can affect bone density and growth. However, the systemic administration of β-adrenergic agents can produce a variety of unwanted side effects. β-adrenergic antagonists, for example, can cause bronchoconstriction, hypoglycemia, heart failure, and CNS effects such as nausea, nightmares, insomnia and depression, dizziness, inability to get or maintain an erection (impotence), cold arms, hands, legs, or feet due to poor blood flow to these areas, slow heart rate, shortness of breath, and wheezing in people with asthma.

These adverse sides can in some cases limit the potential use of β-adrenergic agents in treating bone diseases.

By localizing β-adrenergic agents to bone, the subject bone-targeted delivery of β-adrenergic agents can reduce harmful or undesirable effects of the parent β-adrenergic agent. Because relatively higher doses can be delivered to the bone this way, it may also reduce the effective doses of β-adrenergic agent required for treatment, further reducing undesirable side effects. In addition, the bone-targeting moiety may itself be an agent that affects bone metabolism, including bone resorption and formation. In those embodiments, the combination of β-adrenergic agent and bone-targeting moiety may result in an additive or synergistic effect.

To further illustrate, the bone-targeted β-adrenergic agents of the present invention include conjugated drugs represented in the general formula (I):

(A)ₘ*(B)ₙ

wherein
A, independently for each occurrence, represents a β-adrenergic agent (agonist or antagonist);
B, independently for each occurrence, represents a bone-targeting moiety;
n and m each independently represent integers of 1 or greater (preferably 1-6, and more preferably 1-2); and
* denotes a covalent or non-covalent interaction associating the β-adrenergic agents A with the bone-targeting moieties B.

In certain embodiments, the associating interaction between A and B moieties can be one that is reversible or metabolized under physiological conditions in which the conjugated drug has been distributed and/or localized to bone and other osteoblast-containing organs or sites in the body. In those embodiments, the dissociation releases A or a prodrug form of A. In other embodiments, the associating interaction between A and B moieties is irreversible, in which case each β-adrenergic agent retains, with respect to its effect on osteoblasts, β-adrenergic activity even when provided in the conjugated drug form.

In those embodiments in which m is 2 or greater, and two different β-adrenergic agents are provided in the drug conjugate, each is preferably of the same category—i.e., each A is an agonist or each A is an antagonist.

In certain preferred embodiments, the conjugated drug is represented in the general formula (II):

A-L-B

wherein, A and B are as defined above, and L is suitably a covalent bond between atoms of A and B, or a covalent linker linking A and B to form the conjugated drug.

While described in more detail below, to further illustrate, the linker group(s) may be an alkylene chain, a polyethylene glycol (PEG) chain, polysuccinic anhydride, poly-L-glutamic acid, poly(ethyleneimine), an oligosaccharide, an amino acid chain, or any other suitable linkage. In certain embodiments, the linker group itself can be stable under physiological conditions, such as an alkylene chain.

In other embodiments, the linker used in the conjugated drug can be metabolized (cleaved) under physiological conditions, such as by an enzyme (e.g., the linkage contains a peptide sequence that is a substrate for a peptidase), or by hydrolysis (e.g., the linkage includes one or more hydrolyzable groups selected from an ester, an amide, a carbamate, a carbonate, a cyclic ketal, a thioester, a thioamide, a thiocarbamate, a thiocarbonate, a xanthate and a phosphate ester). In this way, the linker L is metabolized to release A or a prodrug form of A, though is sufficiently stable to remain intact at least until the conjugate is delivered to the proximity of the targeted osteoblasts. Targeted release of the bone-specific therapeutic agent may be achieved by choosing a linking bond or moiety that is selectively labile under the conditions of the target bone region. Merely to illustrate, acid labile linkers can be which are preferentially cleaved under the low pH environment of the bone. For instance, the linker can be one that undergoes hydrolysis at rate 2, 5, 10, 100 or even 1000 times faster at pHs less than 6 or 5, relative to pH7. As another illustration, the linking bond or moiety may be cleaved enzymatically by an enzyme selectively active in the target region. For instance, the linker may be a pyrophosphate molecule. After the bone-targeting moiety binds to the bone matrix, alkaline phosphatase secreted by osteoblasts can cleave the pyrophosphate link, releasing the β-adrenergic agent proximal to targeted osteoblasts.

In other embodiments, the linker is not metabolized, but neither the linker nor the bone-targeting moiety significantly interferes with the adrenergic activity of A.

In still other embodiments, the drug is represented by the general formula (III) of

A::B

in which: A represents a β-adrenergic agent or prodrug thereof, B represents a bone-targeting moiety; and :: represents an ionic bond between A and B that dissociates under appropriate physiological conditions to release A in the vicinity of targeted osteoblasts.

In yet other embodiments, the bone targeting moieties and β-adrenergic agents are associated via non-covalent interactions of linker pairs, such as represented in the general formula (IV):

[(A-L′]ₙ[B-L″]ₘ

wherein

A, B, n and m are as defined above; and

L′ and L″ independently represents linking groups that non-covalently associate with one other to form the drug conjugate. An example of a suitable L′/L″ pair is biotin and streptavidin.

It may also be desirable to conjugate another therapeutic agent to form a multifunctional (e.g., including bifunctional) drug conjugate, e.g., such as represented by general formula (V):

(A)ₘ*(B)ₙ(T)ₚ

wherein

A, B, n, m and * are as defined above;

T represents a therapeutic agent other than a β-adrenergic agent; and

p is an integer of 1 or greater.

Exemplary therapeutic agents that T can be include estrogens or their equivalents, antiestrogens, calcitonin, bisphosphonates, calcium supplements, cobalamin, pertussis toxin, boron, DHEA and other bone growth factors such as transforming growth factor beta, activin, bone morphogenic protein, (HGH) human growth hormone, (EGF) epithelial growth factor, or (FGF) fibroblast growth factor. For example, an exemplary bifunctional conjugate is one that has the ability to deliver β-adrenergic antagonist to bone as well as another osteogenic agent such as an estrogen.

In preferred embodiments, the conjugated drugs of the present invention have a higher therapeutic index (TI) relative to the β-adrenergic agent itself in the treatment of the bone disease or condition. The “therapeutic index” of a drug refers to the ratio of the concentration at which a therapeutic agent exerts an undesired effect to the concentration at which it exerts a desired effect. A higher therapeutic index is preferable as it provides a greater margin of safety.

As stated above, β-adrenergic antagonists are known to have a variety of adverse side effects in sites other than bone, including, for example, bronchoconstriction, hypoglycemia, heart failure, and CNS effects such as nausea, nightmares, insomnia and depression, dizziness, inability to get or maintain an erection (impotence), cold arms, hands, legs, or feet due to poor blood flow to these areas, slow heart rate, shortness of breath, and wheezing in people with asthma.

By targeting a β-adrenergic antagonist to bone, the conjugates of the present invention may have a higher TI compared to the same but unconjugated r-adrenergic antagonist. The increase in therapeutic index can contribute, to such dosing features as: (1) by specifically delivering a β-adrenergic antagonist to bone, its concentration in a patient's circulation is effectively decreased, leading to reduced adverse effects in other parts of the body; and/or (2) bone-targeted delivery of a β-adrenergic antagonist may reduce the amount of a β-adrenergic antagonist to produce a therapeutically effective result, i.e., a lower dose (moles) of β-adrenergic antagonist is administered.

Accordingly, in one embodiment, with respect to at least one undesirable side effect, the compositions of the present invention may have a therapeutic index for modulating bone density or growth at least 5 times greater than the β-adrenergic agent alone, and more preferably at least 10, 50, 100 or even 1000 times greater. For instance, the therapeutic index of the conjugated drug can be higher with respect to one or more side effects including, for example, nausea, nightmares, insomnia and depression, heart failure, and/or hypoglycemia, dizziness, inability to get or maintain an erection (impotence), cold arms, hands, legs, or feet due to poor blood flow to these areas, slow heart rate, shortness of breath, and wheezing in people with asthma.

In preferred embodiments, the subject bone-targeted β-adrenergic agents have a therapeutic index at least 2 times greater, more preferably at least 5, 10 or even 20 times greater than the β-adrenergic agent alone. β-adrenergic antagonists can especially be used in patients suffering from asthma, chronic bronchitis or emphysema, or patients with worsening or severe heart failure.

In exemplary embodiments, the subject conjugated drugs can be used in the treatment or prevention of such bone diseases as osteoporosis, juvenile osteoporosis, osteogenesis imperfecta, hypercalcemia, hyperparathyroidism, osteomalacia, osteohalisteresis, osteolytic bone disease, osteonecrosis, Paget's disease of bone, bone loss due to rheumatoid arthritis, inflammatory arthritis, osteomyelitis, corticosteroid treatment, periodontal bone loss, skeletal metastasis, bone loss due to cancer, age-related bone loss, osteopenia, and degenerative joint disease, as well as in instances where facilitation of bone repair or replacement is desired such as bone fractures, done defects, plastic surgery, dental and other implantations.

In a specific embodiment, the invention provides compositions and methods relating to the selective β₂ agonists and selective β₂ antagonists.

### II. Definitions

Adrenergic receptors are integral membrane proteins which have been classified into two broad classes, the α and the β-adrenergic receptors. Both types of adrenergic receptors mediate the action of the peripheral sympathetic nervous system upon binding of catecholamines. The binding affinity of adrenergic receptors for these compounds forms one basis of the classification: α receptors tend to bind norepinephrine more strongly than epinephrine and much more strongly than the synthetic compound isoproterenol. The preferred binding affinity of these hormones is reversed for the β receptors. In many tissues, the functional responses, such as smooth muscle contraction, induced by a receptor activation, are opposed to responses induced by β receptor binding.

Subsequently, the functional distinction between α and β receptors was further highlighted and refined by the pharmacological characterization of these receptors from various animal and tissue sources. As a result, α and β-adrenergic receptors were further subdivided into α₁, and α₂ and β₁, β₂, and β₃ subtypes.

The terms “β-adrenergic antagonist” and “beta blockers” each refer to an agent that binds to a β-adrenergic receptor and inhibits the effects of β-adrenergic stimulation.

The term “selective β₂ antagonist” means an active agent having β-adrenergic blocking activity which is selective for β₂-adrenergic receptors.

An “adrenergic agonist” refers to an agent that activates, induces or otherwise increases the signal transduction activity of an adrenergic receptor. Adrenergic agonists may include, but are not limited to proteins, antibodies, small organic molecules or carbohydrates. Examples of β-adrenergic agonists include, but are not limited to, catecholamines and catecholamine analogs, isoproterenol, dopamine, and dobutamine.

The term “selective β₂ agonist” means an active agent having β-adrenergic inducing activity which is selective for β₂-adrenergic receptors.

The term “bone disease” refers to any bone disease, disorder or state which results in or is characterized by loss of health or integrity to bone, and includes unwanted or undesired increases and decreases in bone density, growth and/or formation. Bone disease includes, but is not limited to, osteoporosis, osteopenia, faulty bone formation or resorption, Paget's disease, fractures and broken bones, bone metastasis, osteopetrosis, osteoschlerosis and osteochondrosis. In the case of drug conjugates incorporating β-adrenergic antagonists, exemplary bone diseases which can be treated and/or prevented in accordance with the present invention include bone diseases characterized by a decreased bone mass relative to that of corresponding non-diseased bone, such as osteoporosis, osteopenia and Paget's disease. Drug conjugates incorporating β-adrenergic agonists can be used to treat bone diseases characterized by an increased bone mass relative to that of corresponding non-diseased bone, and include osteopetrosis, osteoschlerosis and osteochondrosis.

The drug conjugates of the present invention can be used for both prevention and treatment of bone diseases. “Prevention” of bone disease includes actively intervening, prior to onset, to prevent the development of disease. “Treatment” of bone disease encompasses actively intervening after onset to slow down, ameliorate symptoms of, or reverse the disease or situation.

As used herein, the terms “associated with” or “association” or “bound to” are meant to refer to attachment, linkage or otherwise diffusional coupling of one component of the conjugate to another. Association of the β-adrenergic agent and bone targeting moiety can be via covalent bonding, hydrogen bonding, metallic bonding, van der Waal's forces, ionic bonding, hydrophobic or hydrophilic forces, adsorption or absorption, chelate type associations, or any combination(s) thereof. Also contemplated within the meaning of “associated with” or “association” or “bound to” are solution or dispersion forces wherein the β-adrenergic antagonist moiety may be dissolved and thus solvated with a solvent.

The terms “covalent linker” refers to a direct bond or group of atoms incorporating and connecting the functional groups of two or more discrete and otherwise separate pharmaceutically active moieties. A “reversible” covalent linker is one which is metabolized (e.g., by enzymatic activity, by hydrolysis, etc) under physiological conditions to generate the active β-adrenergic agent or its prodrug. Preferably, the covalent linker moiety is a substantially linear moiety, and includes no more than 50, atoms, and even more preferably less than 25, or even 10 atoms. Preferred linkers are ones which, when metabolized, generate the pharmaceutically active β-adrenergic agent (or their prodrugs) as discrete and separate chemical entities, and if any byproducts also result, such byproducts are generally inert at the dosing concentration of the drug conjugate.

The term “ED₅₀” means the dose of a drug which produces 50% of its maximum response or effect. Alternatively, the dose produces a pre-determined response in 50% of test subjects or preparations.

### III. Exemplary β-Adrenergic Antagonists

The β-adrenergic antagonists and agonists useful in forming the bone-targeted drug conjugates of the present invention include, but are not limited to, small organic molecules, peptides, proteins, antibodies, and carbohydrates. Preferably, the β-adrenergic agents are selective for the β-adrenergic receptors as compared to α-adrenergic receptors and do not have a significant effect on α-adrenergic receptor activity.

An exemplary class of β-adrenergic antagonist conjugates of the present invention has structures represented in the following generic structure (VI):

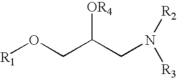

wherein:

R₁, represents: -L-B; a substituted or unsubstituted cyclic or aliphatic moiety; or cyclic moieties including mono- and polycyclic structures which may contain one or more heteroatoms selected from C, N, and O; and

R₂ and R₃ each independently represent: -L-B; hydrogen; or substituted and unsubstituted alkyl;

R₄ represent: -L-B; or hydrogen;

L is suitably a covalent bond or a covalent linker;

B represents a bone-targeting moiety,

at least one of R₁, R₂ and R₃ being -L-B

Another class of beta-blockers that can be used are certain 4-(3-substituted amino-2-hydroxypropoxy)-1,2,5-thiadiazoles. Exemplary thiadiazoles conjugates useful in the present invention have structures represented in the following general structure (VII):

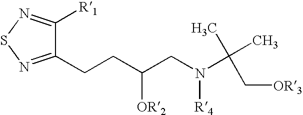

and optically active isomers and pharmacologically acceptable salts thereof, wherein

R′₁ represents: -L-B; hydrogen; a halogen (preferably chloro or bromo); a C₁₋₅ alkyl having either a straight or branched chain (such as methyl, ethyl, propyl, isopropyl, butyl iso-, secondary- or tert-butyl and amyl); a C₂₋₅ alkenyl (such as vinyl, allyl, methallyl and the like); a group having the structure Y—X-Z-, wherein Y is either a straight or branched chain C₁₋₄ alkyl optionally substituted with a phenyl group or a phenyl optionally substituted with one or more halogen atoms (especially chloro, bromo, fluoro), hydroxy, C₁₋₃ alkyl or alkoxy, X is oxygen or sulfur and Z is a methyl or ethyl; a carbamoyl group having the structure R″—HNCO, wherein R″ is a C₁₋₅ alkyl; a C₁₋₅ cycloalkyl (such as cyclopropyl, cyclobutyl, cyclopentyl, cyclohexyl and the like); a C₁₋₄ alkoxy (either a straight or branched chain and including methoxy, ethoxy, propoxy, isopropoxy, butoxy, and pentoxy); a phenyl or substituted phenyl, wherein the substitutes are selected from one or more halogen atoms (preferably chloro or fluoro), C₁₋₃ alkyl or C₁₋₃ alkoxyl; a phenyl-lower alkyl, wherein the phenyl moiety can be unsubstituted or substituted with one or more halogen atoms (preferably chloro, fluoro, or bromo), C₁₋₃ alkyl or C₁₋₃ alkoxyl; an amine having the structure —N(—R′₂)R′₃, wherein R′₂ represents hydrogen, a lower alkyl and a hydroxy-substituted lower alkyl, R′₃ represents hydrogen, a lower alkyl, a hydroxy-substituted lower alkyl and phenyl, or R′₂ and R′₃ can be joined together either directly to give a 3 to 7 membered ring with the nitrogen to which they are attached (e.g., forming aziridinyl, azetidinyl, pyrrolidyl, piperidyl, or a hexahydroazepinyl group), said 3 to 7 membered rings being either unsubstituted or substituted, preferably with one or more lower alkyl and hydroxy-lower alkyl, or alternatively R′₂ and R′₃ can be joined through an oxygen, nitrogen or sulfur atom to form a 5 or 6 membered ring (such as a morpholino, hexahydropyrimidyl, thiazolidinyl, p-thiaiinyl, piperazinyl and the like) optionally substituted by a lower alkyl; or a 5 or 6 membered heterocyclic ring having oxygen, nitrogen or sulfur as the hetero atom (preferably a 2-furyl, 2- or 3-thienyl, 2-pyrryl or an o-, m- or p-pyridyl);

R′₂, R′₃ and R′₄ each independently represent: -L-B; or hydrogen;

L is suitably a covalent bond or a covalent linker;

B represents a bone-targeting moiety,

at least one of R′₁, R′₂, R′₃ and R′₄ being -L-B.

Exemplary β-adrenergic antagonists that be used to form the bone-targeted drug conjugate include the racemic and enantiomeric forms of: Acc 9369, Acebutolol, Alprenolol, AMO-140, Amosulalol, Arotinolol, Atenolol, Befunolol, Betaxolol, Bevantolol, Bisoprolol, Bopindolol, Bucindolol, Bucumolol, Bunitrolol, Bunolol, Bupranolol, Butofilolol, Butoxamine, Capsinolol, Carazolol, Carteolol, Carvedilol, Celiprolol, Cicloprolol, Cloranolol, CP-331684, Diacetolol, Dilevalol, Diprafenone, Ersentilide, Esmolol, Exaprolol, Falintolol, Fr-172516, Hydroxylevobunolol, ICI 118551, Indenolol, IPS 339, Isoxaprolol, ISV-208, L-653328, Labetolol, Levobunolol, Levoprolol, LM-2616, Mepindolol, Metipranolol, Metoprolol, Nadolol, Nebivolol, Nifenalol, Oxprenolol, Pamatolol, Penbutolol, Pindolol, Practolol, Procinolol, Propranolol, SB-226552, Sotalol, SR-58894A, SR-59230A, Tazolol, Tienoxolol, Timolol, Tiprenolol, Toliprolol, Toprol, TZC-5665, UK-1745, Viskenit, Xamoterol, YM-430, and the like.

The β-adrenergic antagonists can be further divided into two groups based on their target selectivities: (1) non-selective β-adrenergic antagonists, which block all three β receptors (for example, propranolol); (2) selective β-adrenergic antagonists, which selectively block one subtype of β receptors. Selective β-adrenergic antagonists may lose selectivity at high doses. Selective β-adrenergic antagonists include selective β1 adrenergic antagonists (for example, atenolol and practolol), selective β2 adrenergic antagonists (for example, butoxamine), and selective β3 adrenergic antagonists. The β-adrenergic antagonists used in the present invention may belong to any of these three groups. However, in certain preferred embodiments, the β-adrenergic antagonist is one that selectively inhibits the β2 adrenergic receptor. Exemplary non-selective β-adrenergic antagonists include nadolol, propranolol, sotalol and timolol. A significant number of compounds having selective β2 antagonist activity suitable for use in this invention are known. These include, but are not limited to, butoxamine, ICI 118,551, H35/25, prenalterol, various 4- and 5-[2-hydroxy-3-(isopropylamino)propoxy] benzimidazoles, 1-(t-butyl-amino-3-ol-2-propyl)oximino-9 fluorene and various 2-(alpha-hydroxyarylmethyl)-3,3-dimethylaziridines. Methods of synthesis, β₂/β₁ selectivity ratios and various biologic and pharmacologic properties of these compounds are known, and reported in for example, J. Pharm. Pharmacol., 1988, 32(9), 659-660; J. Med. Chem., 22(2), 210-214 (1979); J. Med. Chem., 21(1), 68-72 (1978); J. Med. Chem. 20(12), 1657-62 (1977); and Br. J. Pharmacol. 60(3), 357-362 (1977), all of which are herein incorporated by reference. Various other selective β2 adrenergic antagonists are described in U.S. Pat. No. 4,625,586, the entirety of which is incorporated herein.

β₂-adrenergic receptors are found primarily in skeletal and smooth muscle, bone, cartilage, connective tissue, the intestines, lungs, bronchial glands, liver and bladder. β₁-adrenergic receptors are found primarily in the heart, blood vessels and adipose tissue. Accordingly, in certain preferred embodiments, the β-adrenergic antagonist exhibits at least a 10-fold greater potency in inhibiting and/or binding to β₂-receptors relative to β₁-receptors, i.e. have a β₂/β₁ selectivity ratio of at least 5, more preferably at least 10, 50 or even 100. The affinity of various active agents for β₁ and β₂ receptors can be determined by evaluating tissues containing a majority of β₂ receptors (e.g., rabbit ciliary process, rat liver, cat choroid plexus or lung), tissues containing a majority of β₁ receptors (e.g., cat and guinea pig heart), and tissues containing a mixture (e.g., guinea pig trachea). The methods of determining relative binding selectivities for these different types of tissues are extensively disclosed in O'Donnell and Wanstall, Naunyn-Schmiedeberg's Arch. Pharmacol., 308, 183-190 (1979), Nathanson, Science. 204, 843-844 (1979), Nathanson, Life Sciences, 26, 1793-1799 (1980), Minneman et al., Mol. Pharmacol., 15, 21-33 (1979a), and Minneman et al., Journal of Pharmacology and Experimental Therapeutics, 211, 502-508 (1979), all of which are herein incorporated by reference.

In other embodiments, the selectivity of the β-adrenergic antagonist is the consequence of localization of the conjugate and/or localized release of an active antagonist in bone rather than other tissues.

### IV. Exemplary Bone-Targeted Molecules

Suitable bone-targeted molecules are those, when used as a component of the subject drug conjugates result in at least a portion of the conjugate, specifically the β-adrenergic agents of the conjugate being delivered to bone. In other words, suitable bone-targeted molecules, when associated with a therapeutic agent, result in exertion of the pharmacological effects of the agent preferentially on bone, in this case, osteoblasts. The targeting molecules suitably include chemical functionalities exhibiting target specificity, e.g., hormones (e.g., biological response modifiers), and antibodies (e.g., monoclonal or polyclonal antibodies), or antibody fragments having the requisite target specificity, e.g., to specific cell-surface antigens.

The bone-targeted molecules of the present invention may include tetracyclines, calcein, calcitonin, bisphosphonates, chelators, phosphates, polyphosphates, pyrophosphates, phosphonates, diphosphonates, tetraphosphonates, phosphonites, imidodiphosphates, polyaspartic acids, polyglutamic acids, aminophosphosugars, estrogen, peptides known to be associated with mineral phase of bone such as osteonectin, bone sialoprotein and osteopontin, protein with bone mineral binding domains, osteocalcin and osteocalcin peptides, and the like.

The bone-targeted molecules of the present invention may also include peptides of a repetitive acidic amino acid which may work as a carrier for β-adrenergic agents. Examples of suitable small acidic peptides include, but are not limited to, Asp oligopeptides, Glu oligopeptides, gamma-carboxylated Glu (Gla) oligopeptides, as well as peptides comprising a combination of Asp, Glu and Gla. (Asp)₆ or (Glu)₆ are examples of Asp oligopeptides and Glu oligopeptides.

The bone-targeted molecules may also include molecules which themselves affect bone resorption and bone formation rates, such as bisphosphonates, estrogens and other steroids, such as dehydroepiandrosterone (DHEA). These bone-targeted molecules may have affinity for bone and also possess bone growth therapeutic properties and/or result in a synergistic or additive effect with the β-adrenergic agents on bone resorption or formation. Examples of such molecules are bisphosphonates and fluorides.

The following section gives a more in-depth description of some bone-targeted molecules used to form the conjugated drugs of the present invention.

1. Bisphosphonates

Bisphosphonates are synthetic compounds containing two phosphonate groups bound to a central (geminal) carbon. Two characteristics of bisphosphonates make them desirable bone-targeted molecules. First, bisphosphonates have affinity for bone: they are osteoselectively taken up by bone tissue. Bone scanning agents based on the use of some bisphosphonate compounds have been used in the past to achieve desirable high definition bone scans (see e.g., U.S. Pat. No. 4,810,486 to Kelly et al.). Second, bisphosphonates are useful therapeutic agents for bone diseases. They are capable of inhibiting bone loss, believed to act in a manner which hinders the activity of osteoclasts, so that bone loss is diminished. They are useful in treating bone diseases, including Paget's Disease, osteoporosis, rheumatoid arthritis, and osteoarthritis (see e.g., U.S. Pat. No. 5,428,181 to Sugioka et. al).

Bisphosphonates contain two additional chains (R-1 and R-2, respectively) bound to a central geminal carbon. The availability of two side chains allows numerous substitutions and the development of a variety of analogs with different pharmacological properties. The activity varies greatly from compound to compound, the newest bisphosphonates being 5,000 to 10,000 times more active than etidronate, the first bisphosphonate described. The mechanism of action involves:

a) a direct effect on the osteoclast activity;

b) direct and indirect effects on the osteoclast recruitment, the latter mediated by cells of the osteoblastic lineage and involving the production of an inhibitor of osteoclastic recruitment; and

c) a shortening of osteoclast survival by apoptosis. Large amounts of bisphosphonates can also inhibit mineralization through a physicochemical inhibition of crystal growth. The R-1 structure, together with the P—C—P are primarily responsible for binding to bone mineral and for the physicochemical actions of the bisphosphonates. A hydroxyl group at R-1 provides optimal conditions for these actions. The R-2 is responsible for the antiresorptive action of the bisphosphonates and small modifications or conformational restrictions of this part of the molecule result in marked differences in antiresorptive potency. The presence of a nitrogen function in an alkyl chain or in a ring structure in R-2 greatly enhances the antiresorptive potency and specificity of bisphosphonates for bone resorption and most of the newer potent bisphosphonates contain a nitrogen in their structure.

The terms “bisphosphonate” and “bisphosphonates,” as used herein, are meant to also encompass diphosphonates, biphosphonic acids, and diphosphonic acids, as well as salts and derivatives of these materials. The use of a specific nomenclature in referring to the bisphosphonate or bisphosphonates is not meant to limit the scope of the present invention, unless specifically indicated. Non-limiting examples of bisphosphonates useful herein include the following: Alendronic acid, 4-amino-1-hydroxybutylidene-1,1-bisphosphonic acid, Alendronate (also known as alendronate sodium or monosodium trihydrate), 4-amino-1-hydroxybutylidene-1,1-bisphosphonic acid monosodium trihydrate. Alendronic acid and alendronate are described in U.S. Pat. Nos. 4,922,007, to Kieczykowski et al., issued May 1, 1990, and 5,019,651, to Kieczykowski, issued May 28, 1991, both of which are incorporated by reference herein in their entirety. Cycloheptylaminomethylene-1,1-bisphosphonic acid, YM 175, Yarnanouchi (cimadronate), are described in U.S. Pat. No. 4,970,335, to Isomura et al., issued Nov. 13, 1990, which is incorporated by reference herein in its entirety. 1-dichloromethylene-1,1-diphosphonic acid (clodronic acid), and the disodium salt (clodronate, Procter and Gamble), are described in Belgium Patent No. 672,205 (1966) and J. Org. Chem. 32, 4111 (1967), both of which are incorporated by reference herein in their entirety. 1-hydroxy (I-pyrrolidinyl)-propylidene-1,1-bisphosphonic acid (EB-1053). 1-hydroxyethane-I,I-diphosphonic acid (etidronic acid). 1-hydroxy (N-methyl-N-pentylamino)propylidene-1,1bisphosphonic acid, also known as BM-210955, Boehringer-Mannheim (ibandronate), is described in U.S. Pat. No. 4,927,814, issued May 22, 1990, which is incorporated by reference herein in its entirety. 6-amino-1-hydroxyhexylidene-1,1-bisphosphonic acid (nen'dronate). 3-(dimethylamino)-1-hydroxypropylidene-1,1-bisphosphonic acid (olpadronate). 3-amino-1-hydroxypropylidene-I,I-bisphosphonic acid (pamidronate). [2-(2-pyridinyl)ethylidene]-I,I-bisphosphonic acid (piridronate) is described in U.S. Pat. No. 4,761,406, which is incorporated by reference in its entirety. 1-hydroxy (3-pyridinyl)-ethylidene-1,1-bisphosphonic acid (risedronate), (4-chlorophenyl)thlomethane-I,I-disphosphonic acid (tiludronate) as described in U.S. Pat. No. 4,876,248, to Breliere et al., Oct. 24, 1989, which is incorporated by reference herein in its entirety. 1-hydroxy (1H-imidazol yl)ethylidene-1,1-bisphosphonic acid (zoledronate). Preferred are bisphosphonates selected from the group consisting of alendronate, cimadronate, clodronate, tiludronate, etidronate, ibandronate, neridronate, risedronate, piridronate, pamidronate, zoledronate, pharmaceutically acceptable salts or esters thereof, and mixtures thereof. More preferred is alendronate, ibandronate, risedronate, pharmaceutically acceptable salts or esters thereof, and mixtures thereof. More preferred are alendronate, pharmaceutically acceptable salts thereof, and mixtures thereof. Most preferred is alendronate monosodium trihydrate. In other embodiments, other preferred salts are the sodium salt of ibandronate, and risedronate monosodium hemi-pentahydrate (i.e. the 2.5 hydrate of the monosodium salt). See WO02/98354, the content of which is incorporated by reference in its entirety herein.

2. Fluorides

Fluoride is another example of a bi-functional bone-targeted molecule. Fluorides can be taken up by bone, and exert a biphasic action at the level of osteoblasts, on bone mineral, on bone structure and function. Fluorides have been used to treat osteoporosis, alone or in combination with anti-resorptive agents. Rubin and Bilezikian, Endocrinol. Metab. Clin. North. Am., 32: 285-307; Pak et al., Trends Endocrinol. Metab. 6: 229-34.

Fluorides used in the present invention may be in the form of sodium fluoride. The term sodium fluoride refers to sodium fluoride in all its forms (e.g., slow release sodium fluoride, sustained release sodium fluoride). Sustained release sodium fluoride is disclosed in U.S. Pat. No. 4,904,478, the disclosure of which is hereby incorporated by reference. The activity of sodium fluoride is readily determined by those skilled in the art according to biological protocols (e.g., see Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31(1):50-62; Wahner H. W. and Fogelman I., The Evaluation of Osteoporosis: Dual Energy X-Ray Absorptiometry in Clinical Practice, Martin Dunitz Ltd., London 1994, pages 1-296).

3. Small Acidic Peptides

The bone-targeted molecule of the present invention may also be a small acidic peptide. Hydroxyapatite (HA), a major inorganic component and constituent in the matrix of hard tissues such as bone and teeth, may act as a specific site in targeting bone tissue, to which a small acidic peptide may show affinity.

For example, several bone noncollagenous proteins having repeating sequences of acidic amino acids (Asp or Glu) in their structures have an affinity for and tend to bind to hydroxyapatite (HA). Osteopontin and bone sialoprotein, two major noncollagenous proteins in bone, have an Asp and Glu repeating sequence, respectively. Both osteopontin and bone sialoprotein have a strong affinity for and rapidly bind to HA. Therefore, conjugating β-adrenergic antagonist moieties with peptides associated with these and other noncollagenous proteins may be effective in targeting therapeutic delivery of the β-adrenergic antagonist to the bone because of the associated peptides' affinity to HA. (Asp)₆ conjugation may be a particularly effective delivery means because of the high affinity of (Asp)₆ to hydroxyapatite (HA), however (Glu)₆ may be just as effective.

In contrast to bisphosphonate conjugation, acidic peptides used in peptide conjugation tend to degrade in the resorption process, and may show no pharmacological effect. With bisphosphonate conjugation, the treated tissue tends to exhibit some biphosphonate effect. See US 20030129194, the content of which is incorporated by reference in its entirety.

4. Antibody Against Bone-Specific Proteins

The bone-targeted molecule of the present invention may also be an antibody or an antibody fragment. High specificity monoclonal antibodies can be produced by hybridization techniques well known in the art. See, e.g., Kohler et al., 245 Nature 495 (1975); and 6 Eur. J. Immunol. 511 (1976), both of which are incorporated herein by reference. Such antibodies normally may have a highly specific reactivity. Polyclonal antibodies are also suitable for use as the targeting molecule component of the conjugate. However, when the targeting moiety is an antibody, it is most suitably a monoclonal antibody (Mab). Selected monoclonal antibodies are highly specific for a single epitope, making monoclonal antibodies particularly useful as the bone-targeted molecule in the present invention. Suitable antibodies recognize specific cell-surface antigens of bone tissue. Methods for isolating and producing monoclonal or polyclonal antibodies to specific antigens, such as making antibodies to selected target tissue or even to specific target proteins are known. See, e.g., Molecular Cloning, 2nd ed., Sambrook et al., eds., Cold Spring Harbor Lab. Press, 1989, § 18.3 et seq.

5. Metal Ions

The bone-targeted molecule of the present invention may also be a metal ion. Certain metal ions are known to target bone, including, for example, strontium ion. The metal ion may be directly bound to a β-adrenergic antagonist moiety. Alternatively, the metal ion may be linked to a β-adrenergic antagonist moiety via a linker, e.g., an amino acid. For example, it has been disclosed that metal ion-amino acid chelates are capable of targeting tissue site delivery. See, e.g., U.S. Pat. Nos. 4,863,898; 4,176,564; and 4,172,072, each of which is incorporated herein by reference. For example, magnesium-lysine chelates have been shown to target bone. Such chelates are in addition to the polyacidicamino acid conjugates described hereinbefore. The metal ion may be suitably a divalent ion such as Sr²⁺, Zn²⁺, Mg²⁺, Fe²⁺, Cu²⁺, Mn²⁺, Ca²⁺, Cu²⁺, Co²⁺, Cr²⁺ or Mo²⁺.

6. Tracers

The bone-targeted molecule of the present invention may also be a known tracer used to analyze bone metabolism. Such traces include, for example, bone-targeted complexes of technitium-99m, renium 184, rhenium 186. In April 1971, G. Subramanian and J. O. McMee described (Radiology, 99, 192-a) bone scanning agent prepared by reducing pertechnetate TcO4—with stannous chloride in the presence of tripolyphosphate. The resulting labeled complex showed good skeletal uptake but suffered from several disadvantages, the most important of which was a 24-hour delay between injection and scanning (so that high levels of radioactivity were required in order to obtain adequate images), and the instability of the tripolyphosphate with respect to hydrolysis. An intensive search in the 1970's for better phosphate and phosphonate-based bone scanning agents has resulted in a large number of publications and several commercial products. The most widely used compound is methylenediphosphonate (MDP), the complex of which, with Tin and Technetium-99m, is the subject of U.S. Pat. No. 4,032,625. Recent introductions to the market have included hydroxymethylene diphosplionate (RDP), which is the subject of European Patent Application No. 7676; and 1,1-diphosphonopropane-2,3-dicarboxylic acid (DPD), which was described in German O.S. No. 2755874.

Accordingly, in certain preferred embodiments, the subject bone targeting moiety is a phosphonic acid, such as selected from the group consisting of organic di-phosphonic acids, tri-phosphonic acids, tetra-phosphonic acids, tetraminophosphonic acids, and mixtures thereof. Examples of di-phosphonic acids include ethylenehydroxydiphosphonic acid (EHDP), methylenediphosphonic acid (MDP), and aminoethyl-diphosphonic acid (ADEP). Examples of triphosphonic acids include nitrilotri-methylene-phosphonic acid (NTP) and aminotrismethylene-phosphonic acid (AMP). Examples of tetra-phosphonic acids include ethylenediaminetetramethylene-phosphonic acid (EDTMP), nitrilotri-methylene phosphonic acid (NTMP), tetraazacyclo-dodecanetetramethylene phosphonic acid (DOTMP), diethylene-triaminepetnamethylene phosphonic acid (DTPMP).

Tetracycline and its derivatives are another group of tracers with bone affinities. They are routinely used for fluorescent labeling of bone after systemic administration, indicative of their sufficient affinity to mineralized tissue. Suitable tetracycline and derivatives for use in the present invention include, for example, chlortetracycline hydrochloride, demeclocycline hydrochloride, doxycycline, tetracycline, methacycline and oxytetracycline.

Other bone-targeting moieties within the scope of the present compounds are the diphosphonates such as, for example, ethane-1-hydroxy-1,1-diphosphonic acid (EHDP), dichloromethane diphosphonic acid (Cl₂MDP) and 3-amino-1-hydroxypropane-1,1-diphosphonic acid (AHPDP).

7. Heterocyclic Molecules

A series of small, 5-member heterocyclic molecules were discovered to have high bone affinity during routine pharmacokinetics studies. For their structures, see Willson, et al., Med. Chem. Lett., 6:1043 (1996) and Willson et al., Med. Chem. Lett. 6:1047 (1996). Conjugation of a chosen heterocyclic molecule to an estrogenic agent, hexestrol resulted in conjugates with the desired bone affinity. Willson, Id. As such, heterocyclic molecules may be used as the bone-targeted molecule in the present invention.

### V. Linkers

In some embodiments according to the present invention, the β-adrenergic agent and bone targeting moieties are covalently bonded directly to one another, e.g., by forming a suitable covalent linkage through an active group on each moiety. Preferred linker functional groups are primary or secondary amines, hydroxyl groups, carboxylic acid groups or thiol-reactive groups. For instance, an acid group on the moiety may be condensed with an amine, an acid or an alcohol on the other moiety to form the corresponding amide, anhydride or ester, respectively.

In addition to carboxylic acid groups, amine groups, and hydroxyl groups, other suitable active groups for forming linkages between the two, or more, moieties include sulfonyl groups, sulfhydryl groups, thiol and the haloic acid and acid anhydride derivatives of carboxylic acids.

In other embodiments, the moieties in the drug conjugates may be covalently linked to one another through an intermediate linker. The linker advantageously possesses two active groups, one of which is complementary to an active group on the β-adrenergic agent, and the other of which is complementary to an active group on the bone targeting moiety. For example, where the β-adrenergic agent and bone targeting moiety both possess free hydroxyl groups, the linker may suitably be a diacid, which will react with both compounds to form a diether linkage between the two residues. In addition to carboxylic acid groups, amine groups, and hydroxyl groups, other suitable active groups for forming linkages between pharmaceutically active moieties include sulfonyl groups, sulfhydryl groups, and the haloic acid and acid anhydride derivatives of carboxylic acids.

Suitable linkers are set forth in Table 1 below.

| First Pharmaceutically | Second Pharmaceutically | |
|---|---|---|
| Active Compound | Active Compound | |
| Active Group | Active Group | Suitable Linker |
| Amine | Amine | Diacid |
| Amine | Hydroxy | Diacid |
| Hydroxy | Amine | Diacid |
| Hydroxy | Hydroxy | Diacid |
| Acid | Acid | Diamine |
| Acid | Hydroxy | Amino acid, |
| | | hydroxyalkyl acid, |
| | | sulfhydrylalkyl acid |
| Acid | Amine | Amino acid, |
| | | hydroxyalkyl acid, |
| | | sulfhydrylalkyl acid |

Suitable diacid linkers include oxalic, malonic, succinic, glutaric, adipic, pimelic, suberic, azelaic, sebacic, maleic, fumaric, tartaric, phthalic, isophthalic, and terephthalic acids. While diacids are named, the skilled artisan will recognize that in certain circumstances the corresponding acid halides or acid anhydrides (either unilateral or bilateral) are preferred as linker reagents. A preferred anhydride is succinic anhydride. Another preferred anhydride is maleic anhydride. Other anhydrides and/or acid halides may be employed by the skilled artisan to good effect.

Suitable amino acids include γ-butyric acid, 2-aminoacetic acid, 3-aminopropanoic acid, 4-aminobutanoic acid, 5-aminopentanoic acid, 6-aminohexanoic acid, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. Again, the acid group of the suitable amino acids may be converted to the anhydride or acid halide form prior to their use as linker groups. Exemplary linkers are polyglutamic acid or polyaspartic acid, or a linkage group formed by modification of A and/or B and with subsequent bond formation.

Suitable diamines include 1,2-diaminoethane, 1,3-diaminopropane, 1,4-diaminobutane, 1,5-diaminopentane, 1,6-diaminohexane.

Suitable aminoalcohols include 2-hydroxy-1-aminoethane, 3-hydroxy-1-aminoethane, 4-hydroxy-1-aminobutane, 5-hydroxy-1-aminopentane, 6-hydroxy-1-aminohexane.

Suitable hydroxyalkyl acids include 2-hydroxyacetic acid, 3-hydroxypropanoic acid, 4-hydroxybutanoic acid, 5-hydroxypentanoic acid, 5-hydroxyhexanoic acid.

Examples of linkages which can be used include one or more hydrolysable groups selected from the group consisting of an ester, an amide, a carbamate, a carbonate, a cyclic ketal, a thioester, a thioamide, a thiocarbamate, a thiocarbonate, a xanthate, a thiol, a thioester, and a phosphate ester.

In other embodiments, the corticosteroid and other pharmaceutically active moieties may be combined to form a salt.

In still other embodiments, the β-adrenergic agent and bone-targeting moiety are associated through non-covalent binding of bridging linkers. For example, when bone-targeted molecule is a monoclonal antibody, the linker may suitably be a biotin-avidin linkage, using biotin-avidin methodologies known in the art.

Avidin possesses a high affinity for the coenzyme biotin. This is a strong, noncovalent interaction which has been exploited for the conjugation of antibodies to various compounds. The biotin or avidin is suitably coupled to either the β-adrenergic agent or the antibody component. As such, a number of different schemes are possible for linking β-adrenergic agent and antibodies. For example, biotin is suitably linked to the antibody to form a biotinylated antibody complex, while the avidin is suitably linked to the β-adrenergic agent to form an avidin β-adrenergic agent complex. The two complexes are subsequently reacted to form an antibody-biotin-avidin-β-adrenergic agent conjugate.

### VI. Assays for Bone Targeting

The efficacy of bone targeting by the conjugates of the present invention can be measured using any techniques known in the art. This can be achieved by measuring binding of the conjugates of the present invention to bone, or by monitoring bone conditions following administering the compositions of the present invention, as a functional assay.

Binding of the conjugates to bone can be measured in vitro. Specifically, the binding of conjugates of the present invention to hydroxyapatite (mineral component of a bone) can be determined by measuring UV spectra of the conjugates in buffer before and after treatment with hydroxyapatite. A procedure for carrying out this measurement is described in U.S. Pat. No. 6,214,812, the content of which is incorporated by reference herein. Another standard assay that can be used to evaluate bone-targeting is a hydroxyapatite chromatography assay, e.g., where retention time on a hydroxyapatite column can be used to detect agents that are likely to be targeted in vivo to bone.

Alternatively, bone targeting can be measured in vivo. For example, biostribution of the conjugates of the present invention can be measured in rat by complexing the conjugates with a bone tracer, including, for example, ^{99m}Tc, and follow the tracer. Specifically, male rats weighting 160-140 g are injected intravenously via the tail vein. Such measurement is described, for example, in U.S. Pat. No. 6,214,812, mentioned above.

Bone conditions can be monitored using any methods known in the art, including, without limitation, monitoring calcium levels, monitoring bone mass or bone density, monitoring bone turnover, monitoring changes in bone resorption, or monitoring changes in bone characteristics in a biological sample (e.g., blood, plasma, serum, urine, or bone) from the patient following administering the compositions of the present invention. Serum calcium levels can be determined by, for example, atomic absorption spectrophotometry (Cali et al., Clin. Chem., 19:1208-1213 (1973)), chelation with o-cresolphthalein complexone (Harold et al., Am. J. Clin. Pathol., 45:290-296 (1966)), or enzymatically with porcine pancreatic alpha-amylase orthophospholipase D (Kimura et al., Clin. Chem., 42:1202-1205 (1996). Monitoring serum calcium levels is particularly useful in patients with bone conditions related to hyperparathyroidism, renal failure, or hypercalcemia due to malignancy. In such patients, a decrease in calcium levels over the course of treatment indicates that the bone condition is improving.

Bone formation can be monitored by detecting the level of one or more biochemical markers of bone turnover, including osteocalcin, bone specific alkaline phosphatase, and type I C-terminal propeptide (CICP) of type I collagen. For example, the levels of osteocalcin can be detected in serum samples using commercially available immunoassays such as an enzyme-linked immunosorbent assay (ELISA) kit from Immuno Biological Laboratories (Hamburg, Germany) or Diagnostic Systems Laboratories, Inc. (Webster, Tex.) or a radioimmunoassay kit from Phoenix Pharmaceuticals, Inc. (Belmont, Calif.) or Biomedical Technologies Inc. (Stroughton, Mass.). Alternatively, Western blotting can be used. Monitoring osteocalcin levels is particularly useful for patients with a bone condition such as osteoporosis, including osteoporosis resulting from type I diabetes. In osteoporosis patients with high bone turnover, for example, caused by PTH excess, gonadal hormone deficiency, malignancy, or disuse, a decrease in osteocalcin levels over the course of the treatment indicates that the bone condition is improving. Bone specific alkaline phosphatase activity can be monitored in serum samples using commercially available immunoassay kits such as the ALKPHASE-B™ immunoassay kit (Quindel Corp., San Diego, Calif.). CICP, a biochemical indicator of collagen production, can be monitored in serum using an ELISA kit from Quindel Corp. (San Diego, Calif.).

Changes in bone resorption can be monitored by measuring levels of crosslinked collagen such as free deoxypyridinoline and free pyridinoline collagen crosslinks. Free deoxypyridinoline or free pyridinoline can be measured in urine samples using commercially available kits, e.g., an ELISA from Immuno Biological Laboratories (Hamburg, Germany). A decrease in the amount of free deoxypyridinoline or free pyridinoline over the course of the treatment indicates the bone condition is improving.

Bone mass and density also can be monitored in patients treated according to the methods of the invention. Bone mass can be measured in a patient using radiographic imaging techniques such as dual-energy absorptiometry. Bone density can be measured by quantitative computed tomography. An increase in bone mass or density over the course of the treatment indicates that the bone condition is improving in the patient.

### VII. Combinations

The subject drug conjugates can be co-administered, e.g., in the same or different formulation, with a variety of other drugs. For example, the subject β-adrenergic antagonist conjugates can be used as part of a regiment of treatment in which they are combined with other agents that inhibits bone resorption, such as drug which act on osteoclasts. The targets/drugs that are being developed to inhibit bone resorption include but are not limited to the OPG/RANKL/RANK system, cathepsin K inhibitors, vitronectin receptor antagonists, estren, the interleukin-6 and gp130 system, cytokines and growth factors.

Other exemplary agents that can be co-administered with the subject β-adrenergic antagonists include tibolone, new SERMs, androgens, growth hormone, insulin-like growth factor-1 and stontium ranelate.

Exemplary agents that can be co-administered with the subject β-adrenergic agonists include those that promote bone formation, such as lipid-lowering statins and the calcilytic release of PTH.

In certain preferred embodiments, the compositions β-adrenergic agents of the present can be co-administered with a leptin antagonist or agonist, as appropriate. Leptin antagonist, as used herein, refers to a factor which neutralizes or impedes or otherwise reduces the action or effect triggered through activation of a leptin receptor. Such antagonists can include compounds that bind leptin or that bind leptin receptor. Such antagonists can also include compounds that neutralize, impede or otherwise reduce leptin receptor output, that is, intracellular steps in the leptin signaling pathway following binding of leptin to the leptin C) receptor, i.e., downstream events that affect leptin/leptin receptor signaling, that do not occur at the receptor/ligand interaction level. Leptin antagonists may include, but are not limited to proteins, antibodies, small organic molecules or carbohydrates, such as, for example, acetylphenol compounds, antibodies which specifically bind leptin, antibodies which specifically bind leptin receptor, and compounds that comprise soluble leptin receptor polypeptide sequences.

Examples of leptin antagonists are acetylphenols, which are known to be useful as antiobesity and antidiabetic compounds. Since acetylphenols are antagonists of the leptin receptor, they prevent binding of leptin to leptin receptor. Thus, in view of the teachings of the present invention, the compounds would effectively cause an increase in bone mass. For specific structures of acetylphenols which can be used as leptin antagonists, see U.S. Pat. No. 5,859,051.

Leptin antagonists may also include agents, or drugs, which decrease, inhibit, block, abrogate or interfere with binding of leptin to its receptors or extracellular domains thereof; agents which decrease, inhibit, block, abrogate or interfere with leptin production or activation; agents which are antagonists of signals that drive leptin production or synthesis, and agents which prohibit leptin from reaching its receptor, e.g., prohibit leptin from crossing the blood-brain barrier. Such an agent can be any organic molecule that inhibits or prevents the interaction of leptin with its receptor, or leptin production (see, e.g., U.S. Pat. No. 5,866,547). Leptin antagonists include, but are not limited to, anti-leptin antibodies, receptor molecules and derivatives which bind specifically to leptin and prevent leptin from binding to its cognate receptor.

A leptin agonist, as used herein, refers to a factor which activates, induces or otherwise increases the action or effect of triggering a leptin receptor. Such agonists can include compounds that bind leptin or that bind leptin receptor. Such agonists can also include compounds that activate, induce or otherwise increase leptin receptor output, that is, intracellular steps in the leptin signaling pathway following binding of leptin to the leptin receptor, i.e., downstream events that affect leptin/leptin receptor signaling, that do not occur at the receptor/ligand interaction level. Leptin agonists may include, but are not limited to proteins, antibodies, small organic molecules or carbohydrates, such as, for example, leptin, leptin analogs, and antibodies which specifically bind and activate leptin.

Additional leptin antagonists and agonists can be found in U.S. Pat. Nos. 5,972,621; 5,874,535; and 5,912,123, the entirety of all three are incorporated herein.

### VIII. Bone Diseases

Bone diseases which can be treated and/or prevented using β-adrenergic antagonists in accordance with the present invention include bone diseases characterized by a decreased bone mass relative to that of corresponding non-diseased bone, as a result of bone loss. Such bone diseases include both generalized and localized bone loss. The term “generalized bone loss” means bone loss at multiple skeletal sites or throughout the skeletal system. The term “localized bone loss” means bone loss at one or more specific, defined skeletal sites. Generalized boss loss is often associated with osteoporosis. Osteoporosis is most common in post-menopausal women, wherein estrogen production has been greatly diminished. However, osteoporosis can also be steroid-induced (same as glucorticoid therapy below) and has been observed in males due to aging. Osteoporosis can be induced by disease, including, for example, rheumatoid arthritis. Osteoporosis can be induced by secondary causes, including, for example, glucocorticoid therapy (same as steroid-induced above), or it can come about with no identifiable cause, i.e., idiopathic osteoporosis. In the present invention, preferred methods include the treatment or prevention of abnormal bone resorption in osteoporotic humans. Localized bone loss has been associated with periodontal disease, with bone fractures, and with periprosthetic osteolysis (in other words, where bone resorption has occurred in proximity to a prosthetic implant). Generalized or localized bone loss can occur from disuse, which is often a problem for those confined to a bed or a wheelchair, or for those who have an immobilized limb set in a cast or in traction. The methods and compositions of the present invention are useful for treating and or preventing the following conditions or disease states: osteoporosis, which can include post-menopausal osteoporosis, steroid-induced osteoporosis, male osteoporosis, disease-induced osteoporosis, idiopathic osteoporosis; osteopenia, Paget's disease; abnormally increased bone turnover, osteomalacia, renal osteodystrophy, periodontal disease, fracture; and localized bone loss associated with periprosthetic osteolysis.

A critical parameter in diseases of low bone mass is susceptibility to fracture. Since susceptibility to fracture cannot be measure directly, measurements of bone mass or bone mineral density provides an indication of how susceptible a bone is to fracture. Although there is a correlation between low bone mass and increased susceptibility to fracture, there is sometimes discordance which can be attributed to variations in bone geometry and trabecular architecture. In general, bone mass (or bone density or bone volume) and bone geometry are used to obtain a static picture of what a bone looks like, from which the mechanical properties of the bone (e.g., strength, rigidity, and stiffness) are inferred and predictions about risk of fracture can be determined by one of skill in the art. In animal models, histomorphometry measures are favored for analyzing bone mass, geometry, and rate of formation. Rate of resorption is harder to characterize because counts of osteoclast number or surface area are not representative of osteoclast activity. However, a number of serum and urinary markers are becoming available and can be used to detect bone breakdown products. For example, Bone Resorption kit Osteomark® from Biohealth Diagnostics measures urinary cross-linked N-telopeptides, NTx, which is released into the bloodstream during bone breakdown (resorption). The mechanical properties of bone, such as, but not limited to, strength in tension compression and bending, stiffness, and maximal load, can be directly measured. Bone mass and bone geometry can be determined by methods such as, but not limited to, single and dual photon absorptiometry (SPA and DPA), single and dual X-ray absorptiometry (SXA and DXA), quantitative computed tomography (QCT), ultrasound (US) and magnetic resonance imaging (MRI) (see, e.g., Guglielmi et al., 1995, Eur Radiol. 5(2): 129-39).

The bone-targeted β-adrenergic agonists of the present invention can be used as part of the treatment of bone diseases characterized by an increased bone mass relative to that of corresponding non-diseased bone. Exemplary disorders include, but are not limited to, osteopetrosis, osteosclerosis and osteochondrosis.

Bone-targeted β-adrenergic agonists can be used to treat diffuse idiopathic skeletal hyperostosis (dish), a disorder of unknown cause characterized by excessive bone formation at skeletal sites subject to normal or abnormal stresses, generally where tendons and ligaments attach to bone. The spine is the predominant site of involvement, although extraspinal sites may also be affected. Some patients may develop ossification after surgery or in response to coexistent diseases, such as rheumatoid arthritis. This disease is also known by other names, including spondylitis ossificans ligamentosa, spondylosis hyperostotica, senile ankylosing hyperostosis of the spine, Forestier's disease, spondylosis deformans and vertebral osteophytosis. Rheumatoid arthritis and DISH (RA/DISH) can coexist in the same patient.

The subject bone-targeted β-adrenergic agonists can also be used in the treatment of hyperostosis, an excessive growth of bone, which may lead to formation of a mass projecting from a normal bone (exostosis). This abnormality may be seen in numerous musculoskeletal disorders.

A widespread form of hyperostosis characterized by flowing calcification and ossification of vertebral bodies occurs in diffuse idiopathic skeletal hyperostosis DISH. Radiographic abnormalities are observed most commonly in the thoracic spine. In this disease, calcification and ossification may lead to the presence of a radiodense shield in front of the vertebral column. Enthesophytes are frequently seen on various bone surfaces.

Calvarial hyperostosis occurs in various pathologic conditions, including Paget's disease, hyperostosis frontalis interna, frontometaphyseal dysplasia, fibrous dysplasia, anaemia, craniodiaphyseal dysplasia and skeletal metastasis.

Endosteal hyperostosis has three subtypes: van Buchem's syndrome, sclerosteosis and Worth's syndrome. In Van Buchem's syndrome, severe enlargement of the mandible, cranial nerve involvement, a prominent forehead and widened nasal bridge, periosteal excrescences in the tubular bones, osteosclerotic and enlarged ribs and clavicles, and increased radiodensity of the spine are characteristic. In sclerosteosis patients may have excessive height and weight, peculiar facies, hypertelorism, deafness, facial palsy, syndactyly of fingers, absent or dysplastic nails, and radial deviation of the terminal phalanges. On radiographs a progressive marked hyperostosis of the skull and mandible is seen. In Worth's syndrome, enlargement of the jaw and the presence of a palatal mass (torus palatinus) are important clinical signs. Radiographically, cortical thickening in the tubular bones without expansion or abnormal modeling is observed.

Infantile cortical hyperostosis, also known as Caffey's disease, is characterized by soft tissue nodules, periostitis and hyperostoses. Bones (mandible, clavicle, scapula, ribs, tubular bones) and adjacent fasciae, muscles and connective tissues are affected. The most prominent feature of the disease, cortical hyperostosis, begins as a soft tissue swelling directly contiguous to the bone cortex and may lead to doubling or tripling of the normal width of the bone. Destructive lesions of the skull or tubular bones have also been identified.

Sternocostoclavicular hyperostosis is characterized by distinctive bone overgrowth and soft tissue ossification of the clavicle, anterior portion of the upper ribs and sternum. Bone overgrowth may lead to occlusion of the subclavian veins. The major radiographic abnormalities are seen in the anterior and upper portion of the chest wall and vertebral column. Spinal outgrowths may be seen that resemble those of ankylosing spondylitis, diffuse idiopathic skeletal hyperostosis or psoriatic spondylitis.

Vitamin A intoxication and long-term use of isotretinoin have also been associated with skeletal hyperostosis (see hypervitaminosis A).

Various groups of disorders characterized by hyperostosis, osteitis and skin lesions have been termed the SAPHO syndrome. This term also encompasses sternocostoclavicular hyperostosis, arthro-osteitis associated with pustulosis palmaris et plantaris, and arthro-osteitis associated with severe acne. Bone sclerosis is a dominant radiographic abnormality.

In other embodiments, the bone-targeted β-adrenergic antagonists and agonists of the present invention can be used to promote or inhibit bone in-growth into a prosthesis.

Bone-targeted β-adrenergic agonists can be used further to promote union of an area of non-union fracture, promote healing of non-healing wounds, and promoting the integration of dental implants into bone.

The invention also encompasses bone diseases not related to bone mass. For example, the present invention includes, but is not limited to, diseases of altered mineral content, abnormal matrix compounds (e.g., collagen), or abnormal local outgrowths.

### IX. Pharmaceutical Formulations and Methods of Treating Bone Disorders

The compositions of this invention can be formulated and administered to inhibit a variety of bone disease states by any means that produces contact of the active ingredient with the agent's site of action in the body of a mammal. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

Pharmaceutical compositions for use in accordance with the present invention may be formulated in conventional manner using one or more physiologically acceptable carriers or excipients. The therapeutic compositions of the invention can be formulated for a variety of routes of administration, including systemic and topical or localized administration. Techniques and formulations generally may be found in Remington's Pharmaceutical Sciences, Meade Publishing Co., Easton, Pa. For systemic administration, injection is preferred, including intramuscular, intravenous, intraperitoneal, and subcutaneous. For injection, the therapeutic compositions of the invention can be formulated in liquid solutions, preferably in physiologically compatible buffers such as Hank's solution or Ringer's solution. In addition, the therapeutic compositions may be formulated in solid form and redissolved or suspended immediately prior to use. Lyophilized forms are also included.

For oral administration, the therapeutic compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active agent. For buccal administration the therapeutic compositions may take the form of tablets or lozenges formulated in a conventional manner. For administration by inhalation, the compositions for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the therapeutic agents and a suitable powder base such as lactose or starch.

The therapeutic compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

In addition to the formulations described previously, the therapeutic compositions may also be formulated as a depot preparation. Such long acting formulations may be administered by implantation (for example subcutaneously or intramuscularly) or by intramuscular injection. Thus, for example, the therapeutic compositions may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the compositions of the invention are formulated into ointments, salves, gels, or creams as generally known in the art. A wash solution can be used locally to treat an injury or inflammation to accelerate healing. For oral administration, the therapeutic compositions are formulated into conventional oral administration forms such as capsules, tablets, and tonics.

The therapeutic compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

A composition of the present invention can also be formulated as a sustained and/or timed release formulation. Such sustained and/or timed release formulations may be made by sustained release means or delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5,073,543; 5,639,476; 5,354,556; and 5,733,566, the disclosures of which are each incorporated herein by reference. The pharmaceutical compositions of the present invention can be used to provide slow or sustained release of one or more of the active ingredients using, for example, hydropropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable sustained release formulations known to those of ordinary skill in the art, including those described herein, may be readily selected for use with the pharmaceutical compositions of the invention. Thus, single unit dosage forms suitable for oral administration, such as, but not limited to, tablets, capsules, gelcaps, caplets, powders, and the like, that are adapted for sustained release are encompassed by the present invention.

### X. Dosage

The dosage administered will be a therapeutically effective amount of the compound sufficient to result in amelioration of symptoms of the bone disease and will, of course, vary depending upon known factors such as the pharmacodynamic characteristics of the particular active ingredient and its mode and route of administration; age, sex, health and weight of the recipient; nature and extent of symptoms; kind of concurrent treatment, frequency of treatment and the effect desired.

Toxicity and therapeutic efficacy of therapeutic compositions of the present invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapeutic agents which exhibit large therapeutic indices are preferred. While therapeutic compositions that exhibit toxic side effects may be used, care should be taken to design a delivery system that targets such therapeutic agents to the site of affected tissue in order to minimize potential damage to uninfected cells and, thereby, reduce side effects.

The data obtained from cell culture assays and animal studies can be used in formulating a range of dosage for use in humans. The dosage lies preferably within a range of circulating concentrations that include the ED50 with little or no toxicity. The dosage may vary within this range depending upon the dosage form employed and the route of administration utilized. For any agents used in the method of the invention, the therapeutically effective dose can be estimated initially from cell culture assays. A dose may be formulated in animal models to achieve a circulating plasma concentration range that includes the IC₅₀ (i.e., the concentration of the test therapeutic agent which achieves a half-maximal inhibition of symptoms or inhibition of biochemical activity) as determined in cell culture. Such information can be used to more accurately determine useful doses in humans. Levels in plasma may be measured, for example, by high performance liquid chromatography.

It is understood that appropriate doses of small molecule agents depends upon a number of factors known to those or ordinary skill in the art, e.g., a physician. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.

These methods described herein are by no means all-inclusive, and further methods to suit the specific application will be apparent to the ordinary skilled artisan. Moreover, the effective amount of the compositions can be further approximated through analogy to compounds known to exert the desired effect.

The practice of aspects of the present invention may employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986). All patents, patent applications and references cited herein are incorporated in their entirety by reference.

### EXEMPLIFICATION

The invention now being generally described, it will be more readily understood by reference to the following examples, which are included merely for purposes of illustration of certain embodiments and embodiments of the present invention, and are not intended to limit the invention.

The sympathetic nervous system (SNS) is a powerful inhibitor of bone formation by osteoblasts. This function was first uncovered through the analysis of dopamine β-hydroxylase (Dbh)-deficient mice that cannot produce norepinephrine or epinephrine. These mutant mice display, however, multiple endocrine abnormalities that may have masked either the amplitude of the sympathetic regulation of bone remodeling or other roles that the SNS have during bone remodeling. This is why we also had to rely in the past on pharmacological models to study the sympathetic regulation of bone formation. In order to address whether the SNS regulates physiologically other aspects of bone remodeling, mice lacking (β-adrenergic receptor (βAR) are the experimental model of choice since they do not harbor any of the endocrine abnormalities observed in other mouse models of low sympathetic tone (see Table 2 below).

| | WT | b2AR−/− | WT | Dbh−/− |
|---|---|---|---|---|
| Leptin | 2.97 ± 0.68 | 3.61 ± 0.50 | 2.1 ± 0.50 | 3.0 ± 0.40 |
| Insulin | 0.384 ± 0.12 | 0.372 ± 0.16 | 0.341 ± 0.07 | 0.577 ± 0.13 |
| PTH | 136.6 ± 43.9 | 129.1 ± 62.1 | 93.7 ± 15.8 | 99.1 ± 11.7 |
| Cortico- | 203.8 ± 39.1 | 192.3 ± 24.4 | 118.7 ± 43.3 | 238.5 ± 46.8* |
| sterone | | | | |
| (ng/ml) | | | | |

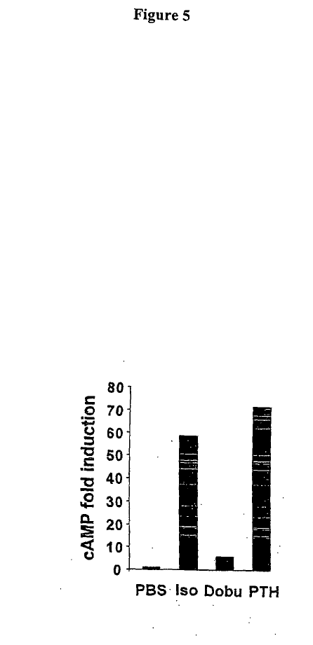

β2AR is the only post-synaptic β-AR whose expression can be detected in osteoblasts. Consistent with this observation, treatment of WT osteoblasts with salbutamol, a β2AR-selective agonist, stimulated cAMP production while treatment with dobutamol, β1AR-selective agonist did not (FIG. 5 and data not shown). Thus, we used Adrb2−/− mice to study how sympathetic signaling affects bone remodeling in adult animals.

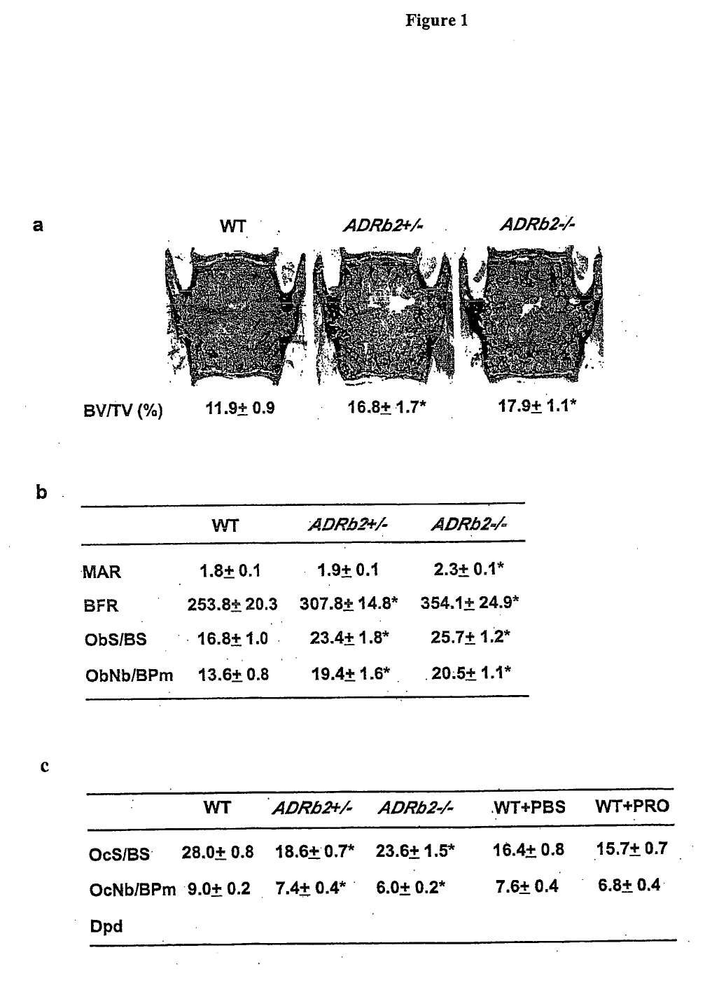

Histological analyses of 6 month-old Adrb2−/− mice revealed a marked increase in bone mass in both genders compared to wildtype (WT) littermates or to mice lacking βAR (FIG. 1a and data not shown). Although expected, the increase in bone mass observed in Adrb2−/− mice was substantially larger than the one observed in Dbh−/− mice most likely because Adrb2−/− mice have none of the endocrine abnormalities plaguing Dbh−/− mice. Histomorphometric analyses showed that Adrb2−/− mice displayed an increase in bone formation as defined by a significant increase in the mineral apposition rate, in the bone formation rate, in the number of osteoblasts and in the surface covered by osteoblasts (FIG. 1b). More unexpected were the evidence of a substantial decrease in bone resorption in Adrb2−/− mice. This was demonstrated histologically by a significant decrease in the surface covered by TRAP-positive multinucleated osteoclasts, suggesting the existence of a defect in osteoclast differentiation, and biochemically by a decrease in urinary elimination of deoxypiridinoline (DpD), a reliable indicator of osteoclast function (FIG. 1c). This ability of sympathetic signaling to regulate in opposite directions bone formation and bone resorption is unique among the known physiological regulators of bone remodeling. This underscores the importance of the sympathetic regulation of bone mass and led us to study how bone resorption is regulated by sympathetic signaling.

First, we analyzed mice lacking only one copy of Adrb2 and compared them to WT mice treated with propranolol, a β-adrenergic receptor antagonist whose administration enhances bone formation in mice. Expression of Adrb2 was first assayed in WT, Adrb2−/− and Adrb2+/− osteoblasts. Adrb2+/− mice, like Adrb2−/−, displayed an increase in bone mass compared to WT mice (FIG. 1a). This was due to both an increase in bone formation and a decrease in bone resorption (FIGS. 1b and 1c). To our knowledge, this high bone mass is the only phenotypic consequence reported so far in the case of haploinsufficiency at the Adrb2 locus. Unlike what is the case in Adrb2+/− mice, propranolol treatment of WT mice did not affect bone resorption in any significant manner. This observation establishes that genetic inactivation of Adrb2 reveals physiological functions of the sympathetic signaling in bone remodeling that could not have been uncovered by pharmacological approaches.

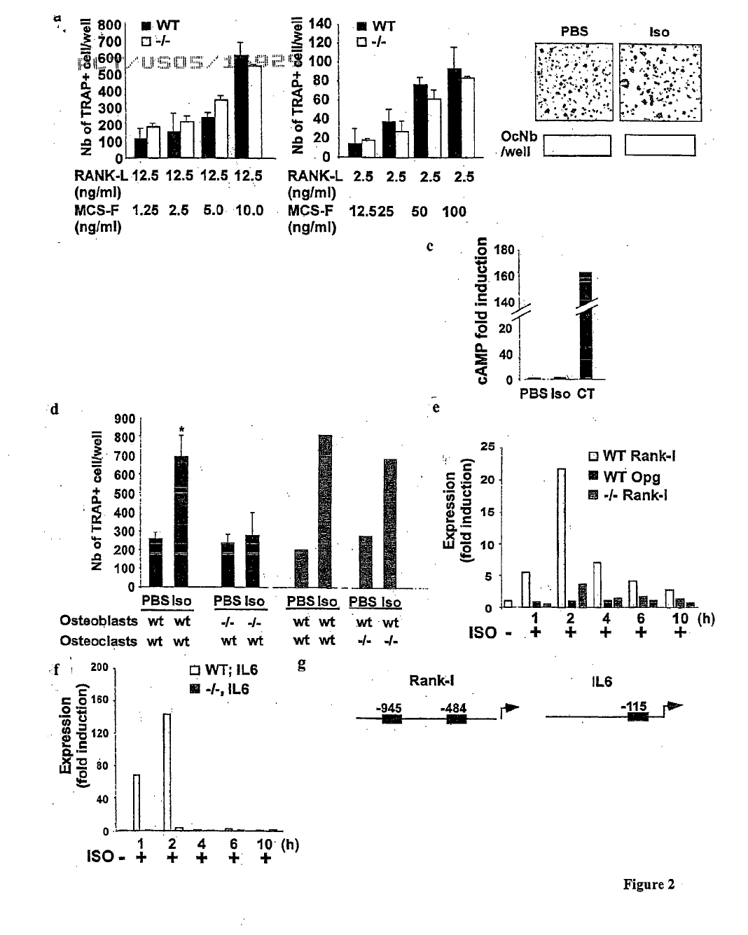

We next asked whether sympathetic signaling affects directly the differentiation or the function of cells of the osteoclast lineage. To test the first hypothesis we used as a bioassay the generation of TRAP-positive multinucleated osteoclasts from the culture of bone marrow macrophages (BMMs) in the presence of RANK-L and M-CSF, two potent osteoclast differentiation factors. Two lines of evidence indicate that sympathetic signaling does not affect directly osteoclast differentiation. First, the number of TRAP-positive multinucleated osteoclasts obtained following treatment of BMMs with limiting doses of RANK-L and M-CSF was similar whether we used WT or Adrb2−/− BMMs at each inductive dose tested, indicating that BMMs could differentiate normally in the absence of Adrb2 (FIG. 2a). Second, addition of isoproterenol, a bAR sympathomimetic in the culture medium during the differentiation of BMMs into osteoclast did not affect the number of TRAP-positive multinucleated osteoclasts that was eventually obtained (FIG. 2b). Osteoclast function was found normal in Adrb2 and ISO-treated WT differentiated osteoclasts. WT or Adrb2 BBMs were differentiated for 2 days with MCS-F and RANK-L, trypsinized and platted on dentine slices for 2 days. Resorption pits were stained with hematoxylin and resorption pit area was quantified (data not shown).

To test whether sympathetic signaling could affect osteoclast function, we treated TRAP-positive multinucleated osteoclasts with isoproterenol. First, unlike what was observed when using osteoblasts, isoproterenol treatment did not induce any significant cAMP production in osteoclasts (FIG. 2c). In contrast, calcitonin (CT), a hormone that transduces its signal through another G-coupled protein receptor also present on osteoclasts induced a robust stimulation of cAMP production. Second, isoproterenol treatment of WT mature osteoclasts did not affect pit formation when testing their ability to resorb bones on dentine slices.

The inability of sympathomimetic to affect in a direct manner osteoclast differentiation or function led us to test whether sympathetic signaling affects bone resorption via its signaling in osteoblasts. To that end we performed co-culture of BMMs and osteoblasts prepared from mouse calvariae. In this assay treatment of osteoblasts with 1,25-(OH)₂ vitamin D₃ leads to the differentiation of BMMs into TRAP-positive multinucleated osteoclasts. When WT osteoblasts and BMMs were used in this co-culture assay, addition of isoproterenol to the culture medium significantly increased the number of TRAP-positive multinucleated osteoclasts (FIG. 2d). Likewise isoproterenol treatment increased osteoclast differentiation when Adrb2−/− rather than WT BBMs were used in the coculture thus confirming that sympathetic signaling does not affect osteoclast progenitor differentiation directly. In contrast, isoproterenol could not enhance osteoclast differentiation when Adrb2−/− osteoblasts were cocultured with WT BMMs suggesting that sympathetic signaling favors bone resorption by stimulating expression in osteoblasts of osteoclast differentiation factors via b2AR.

To test this hypothesis we analyzed the expression in osteoblasts of genes encoding known regulators of osteoclast differentiation following treatment with isoproterenol. In WT osteoblasts isoproterenol increased nearly 20-fold the expression of Rank-l, a gene encoding a secreted molecule required for osteoclast differentiation (FIG. 2e). The induction of Rank-l expression by isoproterenol was not detected when Adrb2−/−osteoblasts were used, indicating that this function of the SNS requires the presence of b2-adrenergic receptors on osteoblasts. Isoproterenol treatment also increased the expression of I16, a cytokine that has been shown to favor osteoclast differentiation (FIG. 2f). These effects of isoproterenol were specific as it did not affect the expression of osteoprotegerin (Opg), a gene that encodes a decoy receptor for RANK-L, of M-CSF or of other interleukins tested such as IL2 or ILI α (data not shown).

That isoproterenol treatment of osteoblasts enhances cAMP production led us to test whether Rank-l and/or I16 expression are regulated by CREB (cAMP response element binding) a transcription factor activated by cAMP signaling pathways. Both Rank-l and I16 promoters contains bona fide CREB binding sites. In chromatin precipitation (ChIP) assays using a phospho-CREB antibody, we showed that CREB bound specifically to Rank-l and I16 promoters. Moreover, in electric mobility shift assays an antibody against phospho-CREB supershifted the protein-DNA complex formed upon incubation of isoproterenol-treated osteoblasts nuclear extracts and a CREB binding site oligonucleotide. To determine whether isoproterenol treatment increases Rank-l and I16 expression via CREB binding to the promoter of these genes, we performed DNA cotransfection experiments in ROS 17/2.8 osteoblastic cells using Rank-l promoter-Luciferase constructs. Altogether these results indicate that sympathetic signaling induces in osteoblast a cascade of signaling events leading to the phosphorylation of CREB and its binding to the promoter of Rank-l and I16-two genes involved in osteoclast differentiation (data not shown).

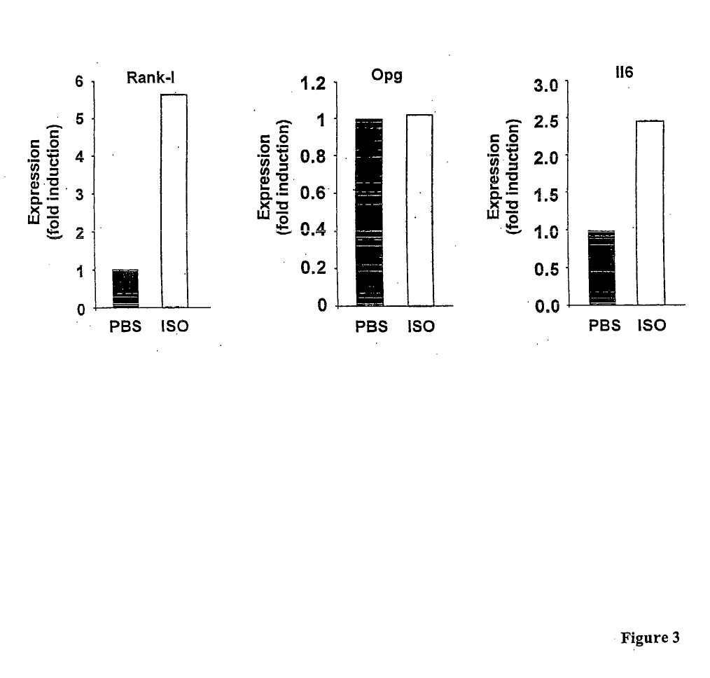

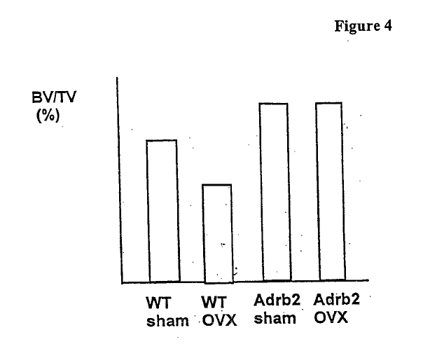

To determine the biological relevance of these findings we performed two experiments. First, we treated WT mice with isoproterenol for 3 weeks and analyzed bone resorption parameters and bone expression of Rank-l and IL6 at the end of this treatment period. Gene expression analysis showed that this treatment increased Rank-l and IL6 expression in bones albeit to a smaller extent than what was observed in vitro while OPG expression was unaffected (FIG. 3). Second, to determine the role that this physiological regulation may have in pathological conditions such as bone loss developing after menopause, we ovariectomized WT and Adrb2−l− mice at one month of age and analyzed them 3 months later. Ovariectomy in WT mice resulted in a 30% decrease in bone mass due to an increase in bone resorption parameters such as osteoclast surface and DpD urinary elimination (FIG. 4). In contrast, osteoclast surface was not increased following ovariectomy in Adrb2−/− mice nor was urinary elimination of Dpd indicating that, in absence of sympathetic tone, bone resorption could not be up-regulated following ovariectomy. The increase in bone formation that persisted together with the absence of any increase in bone resorption explained why Adrb2−/− mice maintained a higher bone mass than WT mice.

### INCORPORATION BY REFERENCE

All publications and patents mentioned herein are hereby incorporated by reference in their entirety as if each individual publication or patent was specifically and individually indicated to be incorporated by reference.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The methods and reagents described herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

It should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A conjugated drug comprising a β-adrenergic agent associated with a bone-targeting moiety so as to increase local delivery and/or efficacy of the β-adrenergic agent to osteoblasts relative to the β-adrenergic agent alone.

2. The conjugated drug of claim 1, wherein said β-adrenergic agent and bone-targeting moiety are covalently associated.

3. The conjugated drug of claim 1, wherein said β-adrenergic agent and bone-targeting moiety are non-covalently associated.

4. The conjugated drug of claim 1, which has a therapeutic index with respect to unwanted side-effects resulting from adrenergic antagonism at least 2 times greater than the therapeutic index of the β-adrenergic agent alone.

5. A conjugated drug that affects bone metabolism, represented in the general formula (I):
(A)ₘ*(B)ₙ
wherein
A, independently for each occurrence, represents a β-adrenergic agent;
B, independently for each occurrence, represents a bone-targeting moiety;
n and m each independently represent integers of 1 or greater; and
* denotes a covalent or non-covalent interaction associating the β-adrenergic agent(s) A with the bone-targeting moieties B.

6. The conjugated drug of claim 5, wherein associating interaction between the A and B moieties is reversible or metabolized under physiological conditions in which the conjugated drug has been distributed and/or localized to bone, the dissociation releasing A or a prodrug form of A.

7. The conjugated drug of claim 5, wherein associating interaction between the A and B moieties is irreversible, said β-adrenergic agent retaining, with respect to osteoblasts, β-adrenergic activity.

8. The conjugated drug of claim 5, which is represented in the general formula (II) A-L-B, wherein, A and B are as defined above, and L is suitably a covalent bond between atoms of A and B, or a covalent linker linking A and B to form the conjugated drug.

9. The conjugated drug of claim 5, which is represented in the general formula (III):
A::B, wherein A and B are as defined above; and :: represents an ionic bond between A and B that dissociates under appropriate physiological conditions to release A in the vicinity of targeted osteoblasts.

10. The conjugated drug of claim 5, which is represented in the general formula (IV):
[(A-L′]ₙ[B-L″]ₘ
wherein
A, B, n and m are as defined above; and
L′ and L″ independently represents linking groups that non-covalently associate with one other to form the drug conjugate.

11. The conjugated drug of claim 5, which is represented in the general formula in the general formula (V):
(A)ₘ*(B)ₙ(T)ₚ
wherein
A, B, n, m and * are as defined above;
T represents a therapeutic agent other than a β-adrenergic agent; and
p is an integer of 1 or greater.

12. The conjugated drug of any of claims 5-11, wherein the β-adrenergic agent is a β-adrenergic antagonist.

13. The conjugated drug of claim 12, wherein the β-adrenergic antagonist is a selective antagonist of the β₂-adrenergic receptor.

14. The conjugated drug of claim 12, wherein the β-adrenergic antagonist is selected from the group consisting of small organic molecules, peptides, proteins, antibodies, and carbohydrates.

15. The conjugated drug of claim 12, represented in the following general structure (VI): 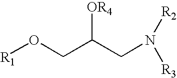
wherein:
R₁, represents: -L-B; a substituted or unsubstituted cyclic or aliphatic moiety; or cyclic moieties including mono- and polycyclic structures which may contain one or more heteroatoms selected from C, N, and O; and
R₂ and R₃ each independently represent: -L-B; hydrogen; or substituted and unsubstituted alkyl;
R₄ represent: -L-B; or hydrogen;
L is suitably a covalent bond or a covalent linker;
B represents a bone-targeting moiety,
at least one of R₁, R₂ and R₃ being -L-B

16. The conjugated drug of claim 12, represented in the following general structure (VII): 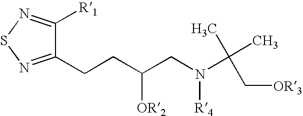
and optically active isomers and pharmacologically acceptable salts thereof, wherein
R′₁ represents: -L-B; hydrogen; a halogen; a C₁₋₅ alkyl; a C₂₋₅ alkenyl; a group having the structure Y—X-Z-, wherein Y is either a straight or branched chain C₁₋₄ alkyl optionally substituted with a phenyl group or a phenyl optionally substituted with one or more halogen atoms, hydroxy, C₁₋₃ alkyl or alkoxy, X is oxygen or sulfur and Z is a methyl or ethyl; a carbamoyl group having the structure R″—HNCO, wherein R″ is a C₁₋₅ alkyl; a C₁₋₅ cycloalkyl; a C₁₋₄ alkoxy; a phenyl or substituted phenyl, wherein the substitutes are selected from one or more halogen atoms, C₁₋₃ alkyl or C₁₋₃ alkoxyl; a phenyl-lower alkyl, wherein the phenyl moiety can be unsubstituted or substituted with one or more halogen atoms, C₁₋₃ alkyl or C₁₋₃ alkoxyl; an amine having the structure —N(—R″₂)R″₃, wherein R″₂ represents hydrogen, a lower alkyl and a hydroxy-substituted lower alkyl, R″₃ represents hydrogen, a lower alkyl, a hydroxy-substituted lower alkyl and phenyl, or R″₂ and R″₃ can be joined together either directly to give a 3 to 7 membered ring with the nitrogen to which they are attached, said 3 to 7 membered rings being either unsubstituted or substituted, preferably with one or more lower alkyl and hydroxy-lower alkyl, or alternatively R′₂ and R′₃ can be joined through an oxygen, nitrogen or sulfur atom to form a 5 or 6 membered ring optionally substituted by a lower alkyl; or a 5 or 6 membered heterocyclic ring having oxygen, nitrogen or sulfur as the hetero atom;
R′₂, R′₃ and R′₄ each independently represent: -L-B; or hydrogen;
L is suitably a covalent bond or a covalent linker;
B represents a bone-targeting moiety,
at least one of R′₁, R′₂, R′₃ and R′₄ being -L-B.

17. The conjugated drug of claim 12, wherein the β-adrenergic antagonists is selected from a group consisting of racemic and enantiomeric forms of: Acc 9369, Acebutolol, Alprenolol, AMO-140, Amosulalol, Arotinolol, Atenolol, Befunolol, Betaxolol, Bevantolol, Bisoprolol, Bopindolol, Bucindolol, Bucumolol, Bunitrolol, Bunolol, Bupranolol, Butofilolol, Butoxamine, Capsinolol, Carazolol, Carteolol, Carvedilol, Celiprolol, Cicloprolol, Cloranolol, CP-331684, Diacetolol, Dilevalol, Diprafenone, Ersentilide, Esmolol, Exaprolol, Falintolol, Fr-172516, Hydroxylevobunolol, ICI-118551, Indenolol, IPS 339, Isoxaprolol, ISV-208, L-653328, Labetolol, Levobunolol, Levoprolol, LM-2616, Mepindolol, Metipranolol, Metoprolol, Nadolol, Nebivolol, Nifenalol, Oxprenolol, Pamatolol, Penbutolol, Pindolol, Practolol, Procinolol, Propranolol, SB-226552, Sotalol, SR-58894A, SR-59230A, Tazolol, Tienoxolol, Timolol, Tiprenolol, Toliprolol, Toprol, TZC-5665, UK-1745, Viskenit, Xamoterol, YM-430, and prodrugs thereof.

18. The conjugated drug of any of claims 5-11, wherein the β-adrenergic agent is a β-adrenergic agonist.

19. The conjugated drug of any of claims 5-18, wherein the bone targeting moiety is selected from the group consisting of: tetracycline, calcein, DHEA, calcitonin, a bisphosphonate, phosphonic acids (such as di-phosphonic acids, tri-phosphonic acids, tetra-phosphonic acids, tetraminophosphonic acids), a pyrophosphate, a chelator, a phosphate, an aminophosphosugar, an estrogen, a peptide, bone sialoprotein and osteopontin, and a protein with bone mineral binding domains.

20. The conjugated drug of claim 19, wherein the bisphosphonate is selected from: alendronate, cimadronate, clodronate, tiludronate, etidronate, ibandronate, neridronate, risedronate, piridronate, pamidronate, tiludronate and zoledronate.

21. The conjugated drug of claim 19, wherein the peptide is a small acidic peptide.

22. The conjugated drug of claim 21, wherein the small acidic peptide is (Asp)₆ or (Glu)₆.

23. The conjugated drug of claim 19, wherein the peptide is associated with associated with mineral phase of bone such as osteonoection, bone sialoprotein or osteopontin.

24. The conjugated drug of claim 8, wherein the linker is cleaved under physiological conditions to release the β-adrenergic agent in the vicinity of osteoblasts.

25. The conjugated drug of claim 24, wherein the linker is cleaved under physiological conditions to release the β-adrenergic agent in the vicinity of osteoblasts.

26. The conjugated drug of claim 25, wherein the linker is a diacid linker, or an acid halide or an acid anhydride thereof.

27. The conjugated drug of claim 24, wherein the linker is an amino acid or peptide linker.

28. The conjugated drug of claim 24, wherein the linker is a diamine.

29. The conjugated drug of claim 24, wherein the linker is an aminoalcohol.

30. The conjugated drug of claim 24, wherein the linker is an hydroxyalkyl acid.

31. The conjugated drug of claim 24, wherein the linker includes a hydrolyzable group selected from the group consisting of an ester, an amide, a carbamate, a carbonate, a cyclic ketal, a thioester, a thioamide, a thiocarbamate, a thiocarbonate, a xanthate, thiol, thioester, and a phosphate ester.

32. The conjugated drug of claim 8, wherein the linker is not cleaved under physiological conditions, and the β-adrenergic agent retains its activity in the conjugated drug form.

33. A method for increasing anabolic bone growth and/or bone density in a mammal, comprising administering to the mammal a therapeutically effective amount of a conjugated drug of any of claims 12-17.

34. The method of claim 33, wherein the mammal has a bone disease characterized by a decreased bone mass compared to that of a corresponding healthy bone.

35. The method of claim 34, wherein the method is part of a treatment or prevention of a bone disease selected from: osteoporosis, osteopenia, Paget's disease, osteomalacia, renal osteodystrophy, periodontal disease, and localized bone loss associated with periprosthetic osteolysis.

36. The method of claim 35, wherein the osteoporosis is post-menopausal osteoporosis, steroid-induced osteoporosis, male osteoporosis, disease-induced osteoporosis, or idiopathic osteoporosis.

37. The method of claim 34, wherein the mammal has a bone disease characterized by gonadal failure-induced bone loss.

38. The method of claim 33, wherein the conjugated drug is co-administered with one or more other agents that inhibit bone resorption.

39. The method of claim 33, wherein the conjugated drug is co-administered with a leptin antagonist.

40. A method for decreasing anabolic bone formation in a mammal, comprising administering to the mammal a therapeutically effective amount of a conjugated drug of claim 18.

41. The method of claim 40, wherein the conjugated drug is co-administered with one or more other agents selected from the group consisting of a leptin, a leptin agonist, and a lipid-lowering statin.

42. The method of claim 40, wherein the method is part of a treatment of a bone disease selected from hyperostosis, osteopetrosis, osteoschlerosis and osteochondrosis.

43. The method of any of claims 33-42, wherein the mammal is a human patient.

44. A packaged pharmaceutical comprising a conjugated drug of any of claims 1-32 in a form suitable for use in human patients, and associated with instructions and/or a label instructing appropriate use and side effects of the conjugated drug in the treatment or prophylaxis of a bone disease.

45. A method for increasing anabolic bone growth and/or bone density in a mammal, comprising administering to the mammal a therapeutically effective amount of at least one β2-selective antagonist.

46. A method for decreasing anabolic bone formation in a mammal, comprising administering to the mammal a therapeutically effective amount of at least one β2-selective agonist.

47. Use of a conjugated drug of any of claims 1-32 in the manufacture of a medicament for increasing anabolic bone growth and/or bone density in a mammal.

48. Use of a conjugated drug of any of claims 1-32 in the manufacture of a medicament for decreasing anabolic bone formation in a mammal.

49. Use of a β2-selective antagonist in the manufacture of a medicament for increasing anabolic bone growth and/or bone density in a mammal.

50. Use of a β2-selective agonist in the manufacture of a medicament for decreasing anabolic bone formation in a mammal.
